(19) United States
(12) Patent Application Publication
(10) Pub. No.: US 2017/0073324 A1
(43) Pub. Date: Mar. 16, 2017

(54) Bifidenone Compositions and Methods of Use

(71) Applicant: Sequoia Sciences, Inc., St. Louis, MO (US)

(72) Inventor: Russell B. Williams, St. Louis, MO (US)

(21) Appl. No.: 14/851,624

(22) Filed: Sep. 11, 2015

(51) Int. Cl. C07D 317/46

(52) U.S. Cl. CPC C07D 317/46

(57) ABSTRACT

A new compound, bifidenone, is provided. Compositions and methods useful for reducing or inhibiting tumor growth including the bifidenone is also provided.

This invention was made with government support under 1R43CA141944-01 awarded by National Institutes of Health. The government has certain rights in the invention.

### BACKGROUND OF THE INVENTION

### Field of the Invention

The present invention relates a new compound, bifidenone, and compositions and methods useful for reducing or inhibiting tumor growth.

### Description of the Related Art

Medicines used by oncologists to treat the majority of solid tumors and various other cancers have not significantly changed in the last twenty years. In fact, nitrogen mustards, antifolates, platinums, nucleoside analogues, taxanes, and anthracyclines are all still first-line or primary therapies despite record increases in R&D spending by the NIH and the pharmaceutical industry. Furthermore, large scale genomics efforts have yielded results that have contradicted expectations of a future full of targeted therapies. For the majority of solid tumors, targeted therapies must still be given along with more traditional non-selective chemotherapies. Thus, there is a need for new chemotherapeutic agents for use as both monotherapies and for combination with new targeted therapies.

The taxanes and vinca alkaloids are first-line chemotherapies that have robustly proven the superior benefits of inhibiting microtubules for increasing overall survival of patients with advanced stage cancer. Newer microtubule inhibitors have further demonstrated the clinical superiority of microtubule inhibitors in breast, prostate, and ovarian cancers and lymphoma. Microtubule inhibitors have withstood dozens of head to head competitive clinical trials unequivocally demonstrating that this is a preferred mechanism of action for antitumor agents. The identification of a new microtubule inhibitor, bifidenone, is reported in the present invention.

### BRIEF SUMMARY OF INVENTION

The present invention provides a compound bifidenone of the following formula (I)

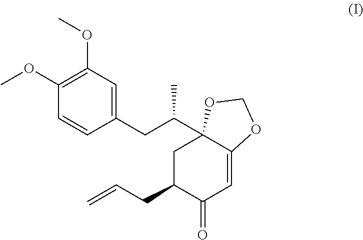 [SMILES: C=CC[C@H]1C[C@]2([C@@H](C)Cc3ccc(OC)c(OC)c3)OCOC2=CC1=O]

Compositions containing the compounds described above and a pharmaceutically acceptable carrier are also contemplated by this invention. As demonstrated herein such compositions are useful in reducing or inhibiting tumor growth or for the treatment of cancer.

In another aspect of the invention, this invention also provides methods for reducing or inhibiting tumor growth comprising contacting the tissue or cell capable of tumor formation with an effective amount of a composition or a compound described above.

In another aspect of the invention, this invention also provides for novel compositions or novel formulations of the compounds of the invention and for methods of administration of these compositions and formulations for reducing or inhibiting tumor growth comprising contacting the tissue or cell capable of tumor formation with an effective amount of a composition or a compound described herein.

In another aspect of the invention, compounds described herein are useful in combination with known anticancer, antitumor, and cytotoxic agents and treatments, including radiation. A preferred combination is with platinum-based anticancer agents.

In another aspect, the present invention provides compositions comprising the compounds of the invention covalently attached to a linker compound that is covalently attached to a small molecule, protein, or antibody and a pharmaceutically acceptable carrier or vehicle. The small molecule, protein, or antibody may be conjugated directly to the compounds of the invention, but more preferably by the means of a linker.

Another aspect of this invention relates to methods of administering the compounds of the invention for the treatment of inflammatory disorders such as gout and other related disorders including but not limited to inflammatory arthritis.

In another aspect of the invention, methods of seed, seedling, or plant treatment with the compounds of the invention are provided that produce new varieties or advantageous or desirable characteristics.

### BRIEF DESCRIPTION OF THE DRAWINGS

FIG. 1. Structure and atom numbering of bifidenone.

FIG. 2. Inactive analogues of bifidenone.

FIG. 3. Inhibition of tubulin polymerization by bifidenone.

FIG. 4. Cell cycle analysis of NCI-H460 lung cancer cells treated with concentrations of bifidenone at 1, 2, or 3× the IC₅₀ value for 6, 18, or 24 h. Cells were then treated with propidium iodide and analyzed using flow cytometry.

FIG. 5. Western blot analysis of NCI-H460 cells treated for 21 h with vehicle, nocodazole, or bifidenone. Nocodazole is known to cause G2/M arrest. Cell lysates were analyzed by western blot using the antibodies indicated on the left. Individual protein bands are designated on the right.

FIG. 6. Caspase assay. Enzyme activity of caspases 3 and 7 was determined using a luminescent assay. NCI-H460 lung cancer cells were treated with varying concentrations of bifidenone or the known antitubulin agents paclitaxel or colchicine. The readout of relative light units is proportional to total caspase 3 and 7 activity.

FIG. 7. Cleavage of PARP and caspase-7 upon treatment with bifidenone.

FIG. 8. Tubulin competition assay. Each graph depicts the amount of unbound compound detected in the presence of varying concentrations of competitors. Known competitors were used as positive controls: podophyllotoxin for the colchicine site (left), vinblastine for the vincristine site (center), and docetaxel for the paclitaxel site (right). Amounts of free compound are expressed as percentages normalized to the amount of free compound detected in the presence of 100 μM known competitor.

### DETAILED DESCRIPTION OF THE INVENTION

Discovery of Bifidenone

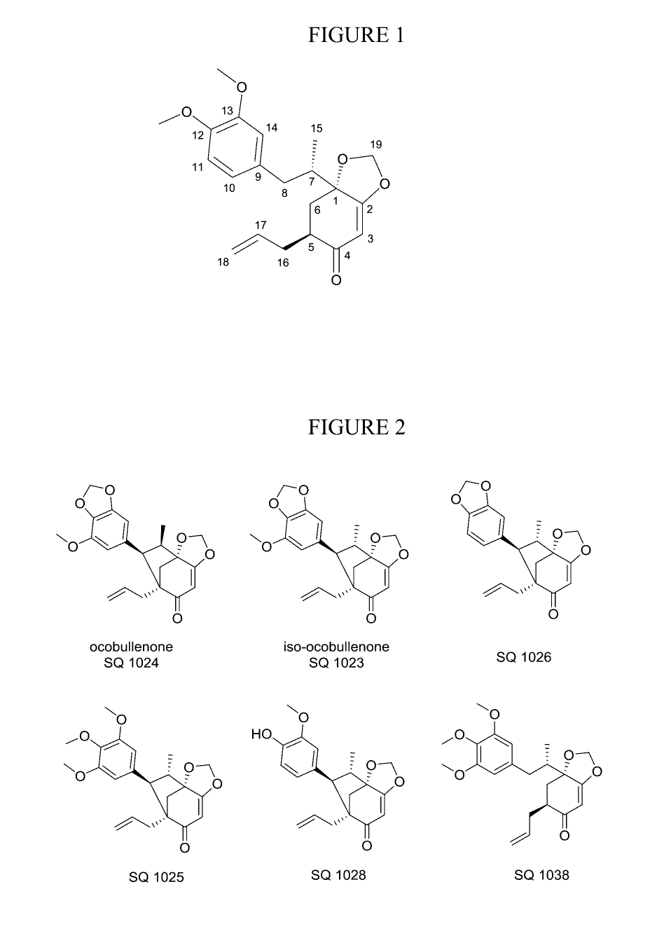

A new compound, bifidenone, the structure and atom numbering of which is shown in FIG. 1, is provided. Bifidenone is an inhibitor of cellular proliferation. Bifidenone was discovered and isolated from a plant of the genus Beilschmiedia (Lauraceae).

The structure of bifidenone was determined using 1D and 2D NMR (Table 1) and HR-ESIMS. The ¹H NMR spectrum closely resembled that of iso-ocobullenone (FIG. 2) (Drewes, S. E., Horn, M. M., Sehlapelo, B. M., Ramesar, N., Field, J. S., Shaw, R. S., Sandor, P., 1995. Iso-ocobullenone and a neolignan ketone from Ocotea bullata bark. Phytochemistry 38, 1505-1508.), a known compound that was re-isolated from this plant specimen. Examination of the 2D NMR spectra, however, indicated that C-8 is a methylene group, and C-5 is a methine. Thus it appeared that the C-8/C-5 ring closure is absent in bifidenone. Furthermore, the aromatic methylenedioxy signals of iso-ocobullenone are replaced in bifidenone with signals for a methoxyl group. Further examination of the 2D HSQC, COSY, and HMBC NMR spectra confirmed the connectivity of the compound in FIG. 1. This molecular formula for this structure (C₂₁H₂₆O₅) is consistent with the observed HR-ESIMS ion (359.1887, [M+H]⁺).

TABLE 1
NMR data for bifidenone (compound 1).

| | | |
|---|---|---|
| Position | ¹³C | ¹H^{a} |

| | | | |
|---|---|---|---|
| 1 | 86.3 | | |
| 2 | 178.1 | | |
| 3 | 98.9 | 5.50 | (s) |
| 4 | 201.4 | | |
| 5 | 43.1 | 2.63 | (m) |
| 6 | 31.8 | 2.13 | (dd, 14.1, 10.0) |
| | | 2.70 | (d, 14.1) |
| 7 | 44.0 | 2.00 | (m) |
| 8 | 36.3 | 2.44 | (dd, 13.8, 11.2) |
| | | 3.11 | (dd, 13.8, 3.5) |
| 9 | 133.6 | | |
| 10 | 113.7 | 6.76 | (d, 1.8) |
| 11 | 150.3 | | |
| 11-OMe | 56.0 | 3.81 | (s) |
| 12 | 149.2 | | |
| 12-OMe | 56.0 | 3.81 | (s) |
| 13 | 112.9 | 6.89 | (d, 7.9) |
| 14 | 121.9 | 6.74 | (dd, 8.2, 1.8) |
| 15 | 14.6 | 0.86 | (d, 7.0) |
| 16 | 37.1 | 2.26 | (ddd, 14.7, 11.4, 8.5) |
| | | 2.90 | (m) |
| 17 | 138.0 | 5.93 | (m) |
| 18 | 117.0 | 5.14 | (br d, 10.3) |
| | | 5.18 | (br d, 17.0) |
| 19 | 102.2 | 5.62 | (s) |
| | | 5.68 | (s) |

^{a}The coupling constants (J) are in parentheses and reported in Hz; chemical shifts are given in ppm.

The relative configuration of bifidenone was determined using the ROESY NMR spectrum and molecular modeling. A ROESY correlation from H-7 to H-16 indicated a 1,3-diaxial arrangement of these two groups, placing them on the same face of the cyclohexenone ring. The compound was then modeled with both configurations of the C-15 methyl group. Only the model with the methyl group down was consistent with the ROESY correlations observed between H-8 and H-15, between H-8 and H-6, and between H-15 and H-19.

To obtain additional bifidenone for antiproliferation assays, the remaining material from the Beilschmiedia specimen was fractionated. During the first isolation it was determined that bifidenone and related compounds are prone to epimerization by the TFA (0.05%) used in the HPLC solvents. Consequently, future isolations were performed without TFA in the semipreparative steps. Ultimately, 79 μg of bifidenone was isolated.

Comparing the structures and antiproliferation activities of bifidenone and other biosynthetically related compounds that were isolated from Beilschmiedia demonstrates that it is not predictable that bifidenone would have such potent activity. The analogues shown in FIG. 2 all had antiproliferation IC₅₀ values>5 μg/mL. Testing the analogues shown in FIG. 2 demonstrates that antiproliferation activity is diminished by an additional ring junction, or by certain alternative substitution patterns of the aromatic ring, or both.

Formula (I) encompass both of the following diastereomers shown below:

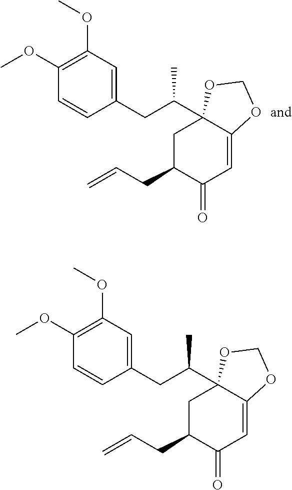 [SMILES: C=CC[C@H]1C[C@]2([C@@H](C)Cc3ccc(OC)c(OC)c3)OCOC2=CC1=O ; C=CC[C@H]1C[C@]2([C@H](C)Cc3ccc(OC)c(OC)c3)OCOC2=CC1=O]

Alternatively, the pair of diastereomers may also be depicted as the following:

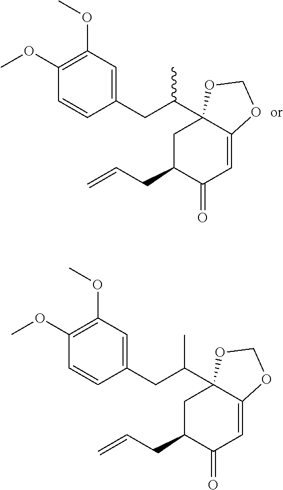 [SMILES: C=CC[C@H]1C[C@]2(C(C)Cc3ccc(OC)c(OC)c3)OCOC2=CC1=O ; C=CC[C@H]1C[C@]2(C(C)Cc3ccc(OC)c(OC)c3)OCOC2=CC1=O]

Furthermore and as described herein, the configuration of the stereogenic centers of the compound of the invention is relative, so the invention (e.g. the compounds of formula (I) or other formula described herein) encompasses the following stereochemical configurations (i.e. enantiomers):

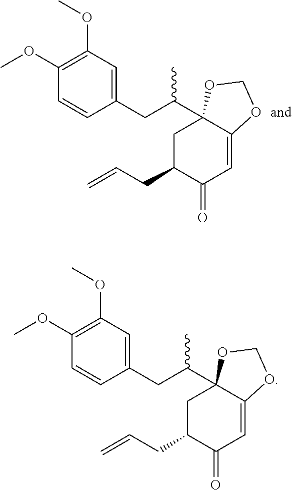

Bifidenone described herein interacts with microtubules. It is thus useful in the treatment of a variety of cancers or other abnormal proliferative diseases, including (but not limited to) carcinoma, hematopoietic tumors, and other tumors. Bifidenone described herein interacts with the colchicine-binding site of tubulin. Such properties may be useful in the treatment of human diseases including gout and familial Mediterranean fever. Bifidenone described herein may also inhibit tumor angiogenesis. Such anti-angiogenesis properties may be useful in the treatment of certain forms of blindness related to retinal vascularization, arthritis, multiple sclerosis, restenosis, and psoriasis. Bifidenone described herein may induce apoptosis. Such apoptosis-inducing properties may be useful in the treatment of human diseases with aberrations in apoptosis including (but not limited to) cancer, viral infections, autoimmune diseases, neurodegenerative disorders, and hematological diseases.

Definitions

“Acceptable carrier” refers to a carrier that is not deleterious to the other ingredients of the composition and is not deleterious to material to which it is to be applied.

“Administration” refers to any means of providing a compound or composition to a subject. Non-limiting examples of administration means include oral, topical, rectal, percutaneous, parenteral injection, intravenous, intravenous infusion, intranasal and inhalation delivery.

“Is one that permits” as it relates to a pharmaceutically acceptable carrier that has characteristics that enable the preparation to be used for a given mode of administration of the composition. For example, pharmaceutically acceptable carriers that permit parenteral administration or intravenous infusion to an animal are liquids that are not injurious or lethal to the animals when so injected. Such carriers often comprise sterile water, which may be supplemented with various solutes to increase solubility. Sterile water or sterile water supplemented with solutes is thus a pharmaceutically acceptable carrier that permits parenteral administration.

“Pharmaceutically acceptable carrier” refers to a carrier that is not deleterious to the other ingredients of the composition and is not deleterious to the human or other animal recipient thereof. In the context of the other ingredients of the composition, “not deleterious” means that the carrier will not react with or degrade the other ingredients or otherwise interfere with their efficacy. Interference with the efficacy of an ingredient does not encompass mere dilution of the ingredient. In the context of the animal host, “not deleterious” means that the carrier is not injurious or lethal to the animal.

“Subject in need thereof” refers to living organism that would benefit from either prevention or reductions in the degree of an abnormal proliferative disease. Subjects may include animals or more specifically, mammals or humans.

Bifidenone described herein may be identified by name 6-allyl-7,7a-dihydro-7a-(1-(3,4-dimethoxyphenyl)propan-2-yl)benzo[d][1,3]dioxol-5(6H)-one instead of or in addition to the structure. The name encompasses any stereoisomeric form, its corresponding enantiomers ((+) and (−) isomers) and diastereomers thereof, and mixtures thereof, and is not limited to any one stereoisomeric form.

Compounds of the Invention

The present invention provides compounds of the following chemical formula (I)

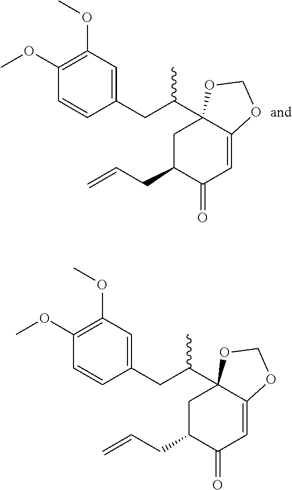

Compositions containing the compounds described above and a pharmaceutically acceptable carrier are also contemplated by this invention. As demonstrated herein such compositions are useful in reducing or inhibiting tumor growth or for the treatment of cancer.

This invention also provides methods for reducing or inhibiting tumor growth comprising contacting the tissue or cell capable of tumor formation with an effective amount of a composition or a compound described above.

In another embodiment, compounds described herein are useful in combination with known anticancer, antitumor, and cytotoxic agents and treatments, including radiation. A preferred combination is with platinum-based anticancer agents.

Such pharmacologic compositions may be formulated in various ways known in the art for administration purposes. Pharmaceutical compositions of the present invention can be prepared by combining an effective amount of the particular compound of this invention, as the active ingredient with one or more pharmaceutically acceptable carriers and delivery vehicles. Numerous pharmaceutically acceptable carriers and delivery vehicles exist that are readily accessible and well-known in the art, which may be employed to generate the preparation desired (i.e. that permit administration of the pharmaceutical composition orally, topically, rectally, percutaneously, by parenteral injection, intravenously, intravenous infusion, intranasally or by inhalation). Representative examples of pharmaceutically acceptable carriers and delivery vehicles include aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene, polyoxypropylene-block polymers, polyethylene glycol and wool fat, and the like. Other constituents, such as aids for taste, color, tableting, and so forth, may be combined with the active ingredient and carrier for any of the many known purposes of such additives. Examples of such additives are discussed below.

The pharmacologic compositions described herein may further be prepared in unitary dosage form for administration orally, percutaneously, by parenteral injection (including subcutaneous, intramuscular, intravenous and intradermal), topically, intranasally, by inhalation, or for application to a medical device, such as an implant, catheter, or other device. In preparing the compositions that permit administration of an oral dosage, for example, any of the pharmaceutically acceptable carriers known in the art may be used, such as water, glycols, oils, alcohols and the like in the case of carriers that permit oral delivery of liquid preparations such as suspensions, syrups, elixirs and solutions. When solid pharmaceutically acceptable carriers are desired that permit oral or rectal administration, starches, sugars, kaolin, lubricants, binders, cellulose and its derivable prodrugs, and disintegrating agents and the like may be used to prepare, for example, powders, pills, capsules and tablets.

For pharmaceutically acceptable carriers that permit parenteral administration, the pharmaceutically acceptable carriers often comprise sterile water, which may be supplemented with various solutes to, for example, increase solubility. Injectable solutions may be prepared in which the pharmaceutically acceptable carrier comprises saline solution, glucose solution, or a mixture thereof, which may include certain well-known anti-oxidants, buffers, bacteriostats, and other solutes that render the formulation isotonic with the blood of the intended patient.

For pharmaceutically acceptable carriers that permit intranasal administration, the pharmaceutically acceptable carriers often comprise poly acrylic acids such as CARBOPOL® 940, polyethylene glycol ethers, nonionic surfactants, a hydrogenated castor oil such as CREMOPHOR® RH40 or CREMOPHOR EL, glycerol, vinylpyrrolidones such as PVP-K90 or PVP-K30, polyethylene glycols such as PEG 1450, benzyl alcohol, edetate sodium, hydroxycellulose, potassium chloride, potassium phosphate, and sodium phosphate. Compositions used for intranasal administration also commonly include benzalkonium chloride as an anti-microbial preservative.

For pharmaceutically acceptable carriers that permit administration by inhalation, the pharmaceutically acceptable carriers often comprise solvent/carrier/water mixtures that are easily dispersed and inhaled via a nebulizer or inhaler. For example, a mixture of ethanol/propylene glycol/water in the ratio of about 85:10:5 (parts ethanol:parts propylene glycol:parts water) can be used to administer the compounds and compositions of the invention via inhalation. Ratios as expressed herein are based on parts by weight.

For pharmaceutically acceptable carriers that permit percutaneous administration, the pharmaceutically acceptable carrier may, optionally, comprise a penetration enhancing agent and/or a wetting agent.

Dosage forms that permit topical or transdermal administration of a compound of this invention include ointments, pastes, creams, lotions, gels, powders, solutions, sprays, inhalants or patches. The active compound or compounds is/are mixed under sterile conditions with a pharmaceutically acceptable carrier and optionally one or more preservatives and/or buffers.

The ointments, pastes, creams and gels may contain, in addition to an active compound or compounds according to the present invention, pharmaceutically acceptable carriers that permit topical or transdermal administration such as animal and vegetable fats, oils, waxes, paraffins, starch, tragacanth, cellulose derivable prodrugs, polyethylene glycols, silicones, bentonites, silicic acid, talc and zinc oxide, or mixtures thereof.

In some cases, the pH of the pharmaceutical formulations contemplated herein may be adjusted with acceptable acids, bases or buffers to enhance the stability of one or more of the active compounds present or their delivery forms.

Still further, in order to prolong the effect of a compound disclosed herein, it may be desirable to slow the absorption of the compound from subcutaneous or intramuscular injection. This may be accomplished using a liquid suspension of crystalline or amorphous material with poor water solubility. The rate of absorption of the compound then depends upon its rate of dissolution, which, in turn, may depend upon crystal size and crystalline form. Alternatively, delayed absorption of a parenterally administered drug form may be accomplished by dissolving or suspending the compound in an oil vehicle.

Injectable depot forms are made, e.g., by forming microencapsule matrices of one or more compounds of the present invention in biodegradable polymers such as polylactide-polyglycolide. Depending upon the ratio of active(s) to polymer and the nature of the particular polymer employed, the rate at which such active(s) is released may be controlled. Examples of other biodegradable polymers include poly(orthoesters) and poly(anhydrides). Depot injectable formulations are also prepared by entrapping the compound in liposomes or microemulsions that are compatible with body tissues.

In another embodiment, an effective amount of a compound of this invention is administered, preferably orally or intravenously, with one or more pharmaceutically acceptable carriers to a subject in need every three weeks. Preferably, an effective amount of a compound of this invention is administered orally or intravenously with one or more pharmaceutically acceptable carriers to a subject in need about every two weeks.

In another embodiment, the present invention provides compositions and methods of administration comprising the compounds of the invention covalently attached to a linker compound that is covalently attached to a small molecule, protein, or antibody and a pharmaceutically acceptable carrier or vehicle. The small molecule, protein or antibody may be conjugated directly to the compounds of the invention, but more preferably by the means of a linker. The examples herein teach how to link the compounds of the invention to linkers and/or moieties used as prodrugs. To those skilled in the art, these compositions may be described as drug conjugates, or more specifically antibody drug conjugates or small molecule drug conjugates. The success of these drug conjugates primarily rests on the cytotoxicity of the drugs, and therefore the invention provide novel compounds to be linked or attached to small molecules, protein or antibodies. Examples of said drug conjugates are described in U.S. Pat. Nos. 7,659,241; 7,662,387, 7,989,434; 8,568,728; and 8,609,105. A further review of drug conjugates more specifically related to the mechanism of the compounds of the invention is described by Vergote et al. (Vergote, I., Leamon, C. P., Vintafolide: a novel targeted therapy for the treatment of folate receptor expressing tumors. Therapeutic Advances in Medical Oncology 7, 206-218.). These references are incorporated herein. Antibodies that have been used in drug-antibody conjugates include but are not limited to anti-CD30 and trastuzumab.

In another embodiment, this invention relates to compositions and methods of administering the compounds of the invention for the treatment of inflammatory disorders such as gout and other related disorders including but not limited to inflammatory arthritis, familial Mediterranean fever or Behcet's disease. Not to be bound by theory, colchicine has a mechanism of action broadly related to the compounds of the invention and has been used to treat gout and other inflammatory disorders, including but not limited to, familial Mediterranean fever, pericarditis, Behcet's disease, and constipation.

In another embodiment, methods of seed, seedling, or plant treatment with the compounds of the invention are provided that produce new varieties or advantageous or desirable characteristics or traits. Not to be bound by theory, colchicine has been used historically to treat seeds to produce desired traits. Since the mechanism of the compounds of the invention is broadly related to colchicine's mechanism, the compounds of the invention may be used to treat seeds, seedlings, or plants to produce certain characteristics or traits.

### EXAMPLES

### Example I

### Isolation of Bifidenone

The plant from which bifidenone was isolated was collected on May 13, 2001 in Nyanga, Gabon at a Kwassa fishing village next to Banio Lagune (S 3 42 59 E 11 0 25). The plant, only identified as Beilschmiedia, was described as a 15 m tall tree with white flowers, green fruit, and fragrant wood. Leaves and stems were collected and dried on site, and then shipped to Sequoia Sciences.

The dried leaves were ground in a blender, and then extracted twice with ethanol-ethyl acetate (1:1). Each extraction was done at room temperature, and consisted of sonication and shaking, followed by gravity filtration. The filtrates were combined and dried to yield 22.1 g of leaf extract. Stems were treated similarly and yielded 8.3 g of stem extract.

Aliquots (1 g) of each extract were subjected to flash chromatography (Phenomenex, STRATA SI-1 Silica, 55 μm, 70 A, 50 g/150 mL Giga Tube). The column was eluted with a stepped gradient consisting of 300 mL each of:

hexanes:ethyl acetate 75:25

hexanes:ethyl acetate 50:50

ethyl acetate

ethyl acetate:MeOH 70:30

ethyl acetate:MeOH 50:50

MeOH:H₂O 50:50 with 1% conc. NH₄OH

Each step was collected as a separate fraction (flash fractions 1-6).

Lipophilic material was removed from flash fraction 2 of the leaf extract using C-18 solid phase extraction (SPE) as follows. Dried flash fraction 2 (477 mg) was dissolved in MeOH and loaded onto the top of a C-18 SPE tube (Phenomenex, STRATA C18-E, 55 μm, 70 A, 5 g/20 mL Giga Tube). The SPE tube was eluted via vacuum manifold using 50% aqueous CH₃CN (100 mL) to yield 302 mg of material (flash fraction 2 SPE). This material inhibited growth of NCI-H460 cells (IC₅₀<20 μg/mL).

Analytical HPLC indicated that the compounds of flash fraction 2 SPE were resolvable, and so it was further separated using semipreparative HPLC. The sample was eluted isocratically using 45% CH₃CN, 0.05% TFA in H₂O over 45 min at 3 mL/min on a C-18 column (Thermo Scientific, HYPERSIL GOLD, 250×10 mm, 5 μm). Ninety-six fractions were collected from 10 to 42 min (0.33 min/fraction). Eight serial collections provided compounds including SQ 1027 and 1028 (15.0 min, 90 μg), SQ 1025 (21.1 min, 113 μg), ocobullenone (28.4 min, 123 μg), iso-ocobullenone (29.4 min, 1760 μg), and SQ 1026 (30.8 min, 152 μg). All of the above were screened for growth inhibition of NCI-H460 along with a peak eluting at 25.9 min (24 μg). The peak eluting at 25.9 min displayed significant activity (IC₅₀<1 μg/mL), while the IC₅₀ for all other fractions and compounds was greater than 1 μg/mL.

Based on the activity of the peak eluting at 25.9 min, the remaining flash fraction 2 SPE material was separated using the semipreparative method described above. Thirty-four serial collections were combined to provide 579 μg of the peak eluting at 25.9 min. Analysis by NMR indicated a mixture, so the 25.9 min peak was further purified using semipreparative HPLC. The sample was eluted from 0 to 5 min with 40% CH₃CN in H₂O, then from 5 to 32 min a gradient from 40% to 43% was applied. Solvents contained 0.05% TFA. The flow rate was 3 mL/min on a perfluorophenyl column (Thermo HYPERSIL KEYSTONE, FLUOPHASE PFP, 250×7.7 mm, 5 μm). Fractions were collected as described above. Five peaks were present in the chromatogram. Four serial collections provided SQ 1039 (mixture) (23.4 min, 177 μg); SQ 1038, 1051, and 1054 (26.3 min, 162 μg); bifidenone and 1052 (28.0 min, 35 μg); SQ 1050 and 1051 (29.3 min, 26 μg); and SQ 1052 and 1053 (31.5 min, 12 μg).

The fraction containing bifidenone was significantly active (IC₅₀=0.1 μg/mL) in the antiproliferation assay against NCI-H460 cells.

To provide more material for additional biological screening, all of the remaining flash fraction 2 from both the leaf and stem extracts was combined (554 mg). The lipophilic material was removed using the SPE method as described above (469 mg). The sample was then separated using preparative HPLC to provide a fraction that was enriched in the peak eluting at 25.9 min. The preparative column was eluted at 20 mL/min using a CH₃CN in H₂O (both containing 0.05% TFA) gradient on a LUNA C-18 column (Phenomenex, 100×21.1 mm, 5 μm, 100 A). The gradient started at 30% CH₃CN from 0 to 2 min then increased from 30% CH₃CN to 49% CH₃CN from 2 to 47 min. Forty fractions were collected from 10 to 50 min (1 min/fraction). The fractions eluting from 32 to 34 min (fractions 23 and 24) were selected based on analytical HPLC for further fractionation.

Semipreparative HPLC was performed isocratically using 45% CH₃CN in H₂O (both containing 0.05% TFA) over a 45 min period at 3 mL/min on a C-18 column (Thermo Scientific, HYPERSIL GOLD, 250×10 mm, 5 μm), as described above. Fifteen serial collections provided 344 μg of crude bifidenone (24.7 min). Finally, the crude bifidenone sample was eluted from 0 to 5 min with 40% CH₃CN in H₂O, and then from 5 to 32 min a gradient from 40% to 43% was applied. The flow rate was 3 mL/min on a perfluorophenyl column (Thermo HYPERSIL KEYSTONE, FLUOPHASE PFP, 250×7.7 mm, 5 μm). TFA was omitted from this final step to prevent epimerization of bifidenone during isolation. Fractions were collected as described above. Six serial collections provided 44 μg of bifidenone (27.1 min).

### Example II

### Inhibition of Cancer Cell Proliferation In Vitro

Bifidenone was tested for its ability to inhibit the proliferation of human cancer cells in vitro as described in the following paragraph. IC₅₀ is defined as the concentration of compound necessary to achieve 50% of the maximum growth inhibition.

For the antiproliferation assay, NCI-H460 (large cell lung carcinoma) or M14 (amelanotic melanoma) cells were grown in RPMI-1640 with 10% FBS supplemented with L-glutamine and HEPES. Cells were seeded into 96-well plates at 5×10² to 5×10⁴ cells/well and allowed to adhere overnight; the medium was then removed. A stock solution of test compound in DMSO was diluted in medium to generate a series of working solutions. Aliquots (100 μL) of the working solutions were then added to the appropriate test wells to expose cells to the final concentrations of compound in a total volume of 100 μL. Seven different concentrations were tested, with 2-5 wells per concentration. Camptothecin was used as a positive control; wells containing vehicle without compound were used as negative controls. Plates were kept for 72 h in a 37° C., 5% CO₂ incubator. After incubation, viable cells were detected with the CELLTITER 96 AQᵤₑₒᵤₛ Non-Radioactive Cell Proliferation Assay (Promega), and IC₅₀ values were determined using GraphPad Prism 5 software.

The IC₅₀ of bifidenone against the M14 cell line is 0.16 μM, and against NCI-H460 is 0.36 μM.

### Example III

### NCI-60 Panel and COMPARE Analysis

Bifidenone was examined for antiproliferation activity against the NCI-60 panel of cancer cell lines. The IC₅₀ values are shown in Table 2. COMPARE analysis was then conducted to compare the activity “fingerprint” of compound 1 to those of known classes of compounds (Zhou, B.-N., Hoch, J. M., Johnson, R. K., Mattern, M. R., Eng, W.-K., Ma, J., Hecht, S. M., Newman, D. J., Kingston, D. G. I., 2000. Use of COMPARE Analysis to Discover New Natural Product Drugs: Isolation of Camptothecin and 9-Methoxycamptothecin from a New Source. Journal of Natural Products 63, 1273-1276.). This analysis indicated that bifidenone acts on microtubules.

TABLE 2
IC₅₀ values for Bifidenone against the NCI 60-cell panel.

| Cell Line | IC₅₀ |
|---|---|
| A498 | 0.46 |
| A549 | 0.26 |
| ACHN | 0.85 |
| BT-549 | 0.18 |
| Caki-1 | 0.10 |
| CCRF-CEM | 0.15 |
| COLO205 | 0.40 |
| DU-145 | 0.84 |
| EKVX | 0.28 |
| HCC-2998 | 1.41 |
| HCT-15 | 0.23 |
| HCT-116 | 0.49 |
| HL-60 | 0.12 |
| HOP-62 | 0.32 |
| HOP-92 | 0.24 |
| Hs578T | 0.21 |
| HT29 | 0.31 |
| IGR-OV1 | 0.42 |
| K-562 | 0.18 |
| KM12 | 0.32 |
| LOX IMVI | 0.39 |
| M14 | 0.20 |
| MALME-3M | 0.09 |
| MCF-7 | 0.17 |
| MDA-MB-231 | 0.26 |
| MDA-MB-435 | 0.07 |
| MDA-MB-468 | 0.30 |
| MOLT-4 | 0.17 |
| NCI-ADR/RES | 0.17 |
| NCI-H23 | 0.61 |
| NCI-H226 | 0.81 |
| NCI-H322M | 0.46 |
| NCI-H460 | 0.32 |
| NCI-H522 | 0.15 |
| OVCAR-3 | 0.17 |
| OVCAR-4 | 0.29 |
| OVCAR-5 | 0.52 |
| OVCAR-8 | 0.47 |
| PC-3 | 0.19 |
| RPMI-8226 | 0.25 |
| RXF-393 | 0.78 |
| SF-268 | 0.22 |
| SF-295 | 0.41 |
| SF-539 | 0.26 |
| SK-MEL-2 | 0.13 |
| SK-MEL-5 | 0.25 |
| SK-MEL-28 | 0.13 |
| SK-OV-3 | 0.27 |
| SN12C | 0.33 |
| SNB-19 | 0.30 |
| SNB-75 | 0.27 |
| SR | 0.08 |
| SW-620 | 0.13 |
| T-47D | 0.25 |
| TK-10 | 0.33 |
| U251 | 0.61 |
| UACC-62 | 0.14 |
| UACC-257 | 0.16 |
| UO-31 | 0.57 |
| 786-0 | 0.32 |

### Example IV

### Microtubule Polymerization

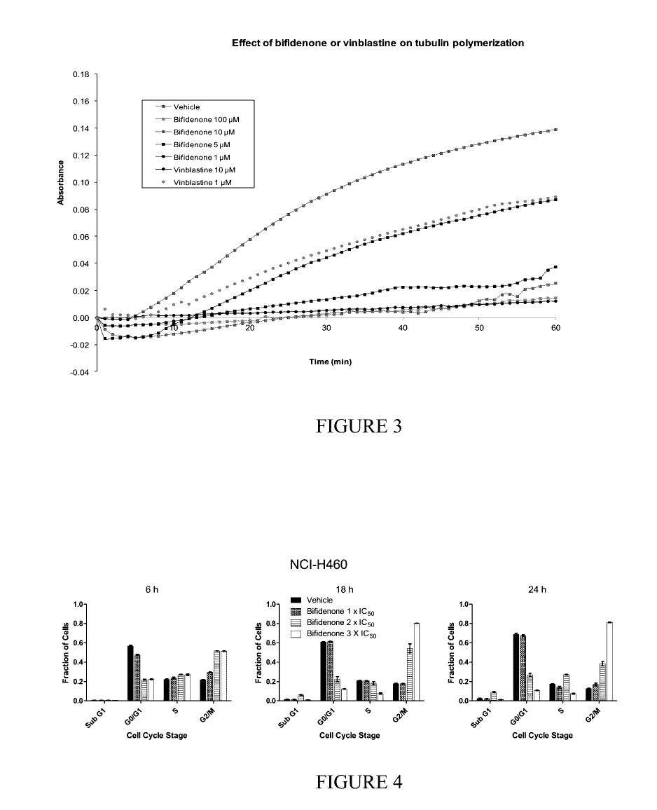

To test the hypothesis that bifidenone inhibits cell proliferation by interfering with microtubule dynamics, it was tested in an in vitro tubulin polymerization assay. Bifidenone inhibits tubulin polymerization in a dose-dependent manner as shown in FIG. 3. FIG. 3 illustrates inhibition of tubulin polymerization by bifidenone. Increased absorbance corresponds to increased polymerization.

### Example V

### G2/M Arrest and Apoptosis

Antitubulin agents typically interfere with mitosis by disrupting the microtubule dynamics necessary for cell division. Cells unable to complete mitosis eventually undergo apoptosis. Consistent with the hypothesis that bifidenone interferes with microtubule dynamics, cell cycle analysis indicated that bifidenone causes arrest in the G2/M phase of the cell cycle, shown in FIG. 4. FIG. 4 shows cell cycle analysis of NCI-H460 lung cancer cells treated with concentrations of bifidenone at 1, 2, or 3× the IC₅₀ value for 6, 18, or 24 h. Cells were then treated with propidium iodide and analyzed using flow cytometry.

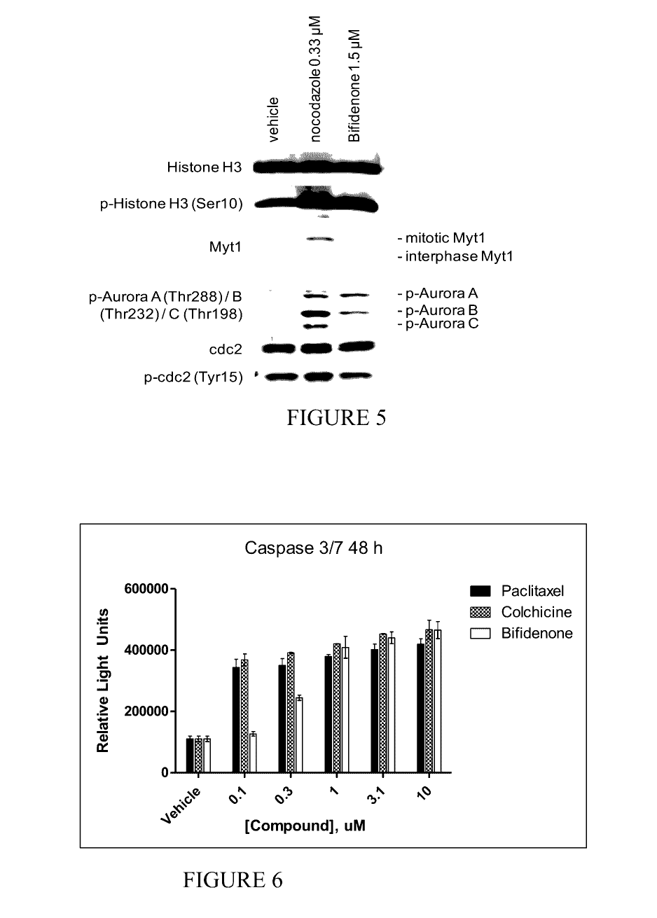

In addition, cells treated with bifidenone display protein expression and phosphorylation patterns consistent with G2/M arrest, including increased phosphorylation of Histone H3 at serine 10, hyperphosphorylation of Myt1, increased phosphorylation of the Aurora A/B/C kinases, and stable or decreased phosphorylation of cdc2 at tyrosine 15 (FIG. 5). FIG. 5 shows western blot analysis of NCI-H460 cells treated for 21 h with vehicle, nocodazole, or bifidenone. Nocodazole is known to cause G2/M arrest. Cell lysates were analyzed by western blot using the antibodies indicated on the left. Individual protein bands are designated on the right.

An apoptosis assay that measures enzymatic activity of caspases 3 and 7 indicated that cells treated with bifidenone also undergo apoptosis (FIG. 6). FIG. 6 illustrates Caspase assay. Enzyme activity of caspases 3 and 7 was determined using a luminescent assay. NCI-H460 lung cancer cells were treated with varying concentrations of bifidenone or the known antitubulin agents paclitaxel or colchicine. The readout of relative light units is proportional to total caspase 3 and 7 activity.

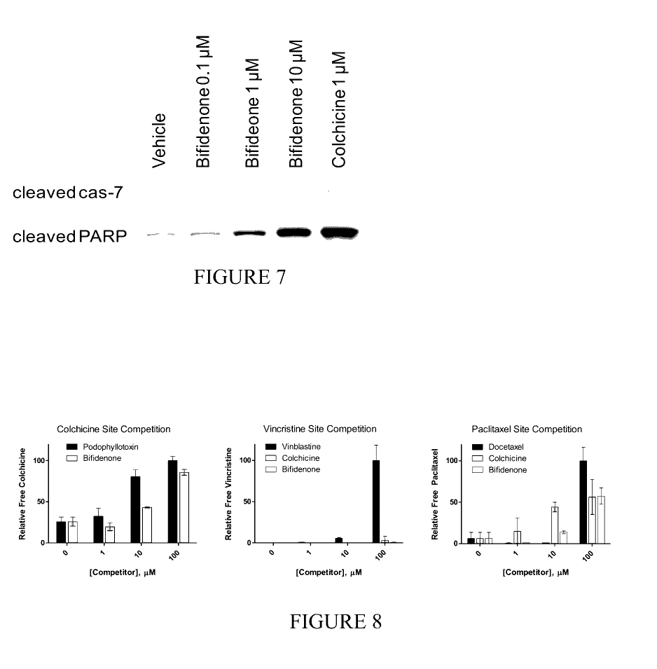

Consistent with apoptosis detected with the caspase 3/7 enzymatic assay, Western blots confirmed that cleavage of caspase-7 and PARP occurred upon treatment with bifidenone (FIG. 7). FIG. 7 shows cleavage of PARP and caspase-7 upon treatment with bifidenone

### Example VI

### Colchicine Binding Site

The in vivo and in vitro data described above indicated that bifidenone and its analogs inhibit cell proliferation through interaction with tubulin. In order to determine the binding site of bifidenone on tubulin, a published ultrafiltration-based competition assay was used. In this assay, tubulin is allowed to bind with a known ligand, and then free ligand is separated by ultrafiltration and analyzed by LC/MS. In the presence of a competitor, the concentration of free ligand increases. Using this assay, it was demonstrated that bifidenone competes for the colchicine binding site, and weakly for the paclitaxel binding site, but not for the vincristine binding site of tubulin (FIG. 8). FIG. 8 illustrates tubulin competition assay. Each graph depicts the amount of unbound compound detected in the presence of varying concentrations of competitors. Known competitors were used as positive controls: podophyllotoxin for the colchicine site (left), vinblastine for the vincristine site (center), and docetaxel for the paclitaxel site (right). Amounts of free compound are expressed as percentages normalized to the amount of free compound detected in the presence of 100 μM known competitor.

### Example VII

### Pharmacokinetics

The bioavailabilities of certain compounds were examined in mice. Administration of the compounds was performed by intraperitoneal (IP) injection using a vehicle as known to those skilled in the art. To measure compound concentrations in plasma, blood is collected by cardiac puncture at a specified time after dosing. It is then centrifuged in a plasma separator tube with NaEDTA/NaF as the anticoagulant, and the resulting plasma is stored at −80° C. For analysis, an internal standard is added to a 200 μL aliquot of plasma, and the aliquot is extracted with ethyl acetate. The ethyl acetate extract is analyzed by LC/MS and quantitated using a standard curve prepared by adding known amounts of test compound and internal standard to 200 μL aliquots of blank mouse plasma.

Many vehicles can be used to examine bioavailability. Prior to administration, each vehicle was optimized based on compound solubility according to the formulation research conducted by Uckun et at (Uckun, F. M., Tibbles, H., Erbeck, D., Venkatachalam, T. K., Qazi, S., 2007). In vivo pharmacokinetics and toxicity of a novel hydrophilic oral formulation of the potent non-nucleoside reverse transcriptase inhibitor compound N′-[2-(2-thiophene)ethyl]-N′-[2-(5-bromopyridyl)]-thiourea (HI-443). Arzneimittelforschung 57, 218-226.). Based on this publication, vehicles containing approximate ratios of 2:1:1 of propylene glycol: TWEEN-20:PEG400 (TWEEN-20 being a common emulsifier used in formulations and food products) and less than 5% ethanol upon administration exhibit good solubility properties and increase serum bioavailability. During these experiments, PEG400 demonstrated a critical role in serum bioavailability. The concentration of PEG400 is modulated depending upon solubility of the compounds and the amount of water phase added.

Mouse plasma concentrations (1 h post-injection) of bifidenone greater than its IC₅₀ against NCI-H460 human lung cancer cells (360 nM) were initially achieved with a 40 mg/kg injection in a vehicle consisting of 40% propylene glycol, 20% TWEEN-20, 20% PEG400, 20% ethanol (v/v). Plasma concentrations were not diminished when this formulation was diluted with up to 20% water. Additional experiments demonstrated that plasma levels were diminished when propylene glycol was decreased to 25%, but that plasma levels increased or were not diminished when propylene glycol was increased to 45% and TWEEN-20 was decreased to <3%. Using the optimized bifidenone vehicle, consisting of 40.5% propylene glycol, 2.7% TWEEN-20, 31.5% PEG400, 15.3% ethanol, 10% water (v/v), injections of 40 mg/kg resulted in bifidenone plasma concentrations>6 times the IC₅₀ at 30 min, >3 times the IC₅₀ at 1 h, and greater than the IC₅₀ at 3 h. With larger injections (75-100 mg/kg), plasma concentrations were >4 times the IC₅₀ at 6 h.

### Example VIII

### Synthesis of Bifidenone (1)

Preparation of Intermediate I.

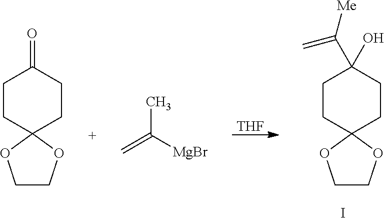

To a three-neck flask equipped with a mechanical stirrer, an addition funnel and a thermometer, was charged a solution of isopropenyl magnesium bromide (0.5M solution in THF, 1.6 L, 0.80 mol) by cannula under a nitrogen atmosphere. The solution was cooled to 0-5° C. A solution of 1,4-dioxaspiro[4.5]decan-8-one (100 g, 0.64 mol) in THF (600 mL) was slowly added by an addition funnel over 2 h while keeping the temperature below 5° C. After the addition, the reaction mixture continued to stir for 2 h. The reaction was quenched by saturated ammonium chloride (200 mL) and extracted with ethyl acetate (2 L). The organic phase was washed with brine, dried (MgSO₄), and concentrated in vacuo to provide I as a brown oil (123 g, 100%). The product was used without further purification. ¹H NMR (300 MHz, CDCl₃): δ 5.30 (s, 1H), 5.05 (s, 1H), 4.00-3.91 (m, 4H), 2.04-1.95 (m, 4H), 1.81 (s, 3H), 1.68 (d, J=12.9 Hz, 4H), 1.17 (s, 1H).

Preparation of Intermediate II.

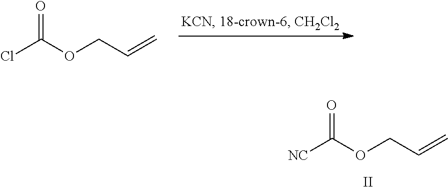

A mixture of allyl chloroformate (48 g, 0.40 mol), KCN (28.8 g, 0.44 mol) and 18-crown-6 (400 mg) in CH₂Cl₂ (400 mL) was stirred under a nitrogen atmosphere at room temperature for 24 h. The reaction mixture was filtered and washed with CH₂Cl₂ (50 mL). The filtrate was distilled to remove CH₂Cl₂ at 60° C. (oil bath temperature). The oil bath temperature was heated to 100° C. The product was distilled under reduced pressure (120 mmHg) to provide II (25 g, 57%) as a colorless oil. ¹H NMR (500 MHz, CDCl₃): δ 6.01-5.93 (m, 1H), 5.44 (dd, J=18.5, 3.0 Hz, 1H), 5.35 (dd, J=10.5, 2.5 Hz, 1H), 4.80 (dd, J=6.5, 5.5 Hz, 2H).

Procedure: Illustrated with Preparation of Bifidenone.

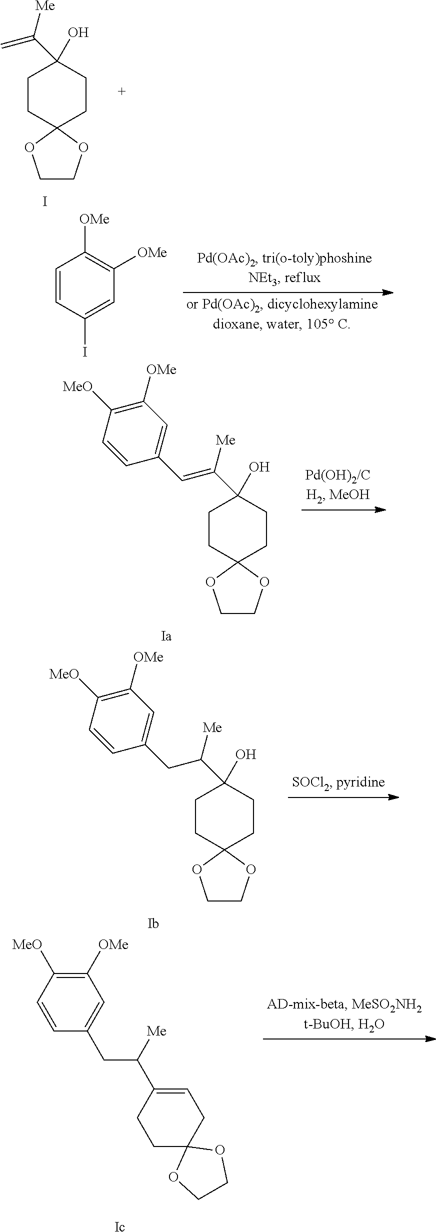

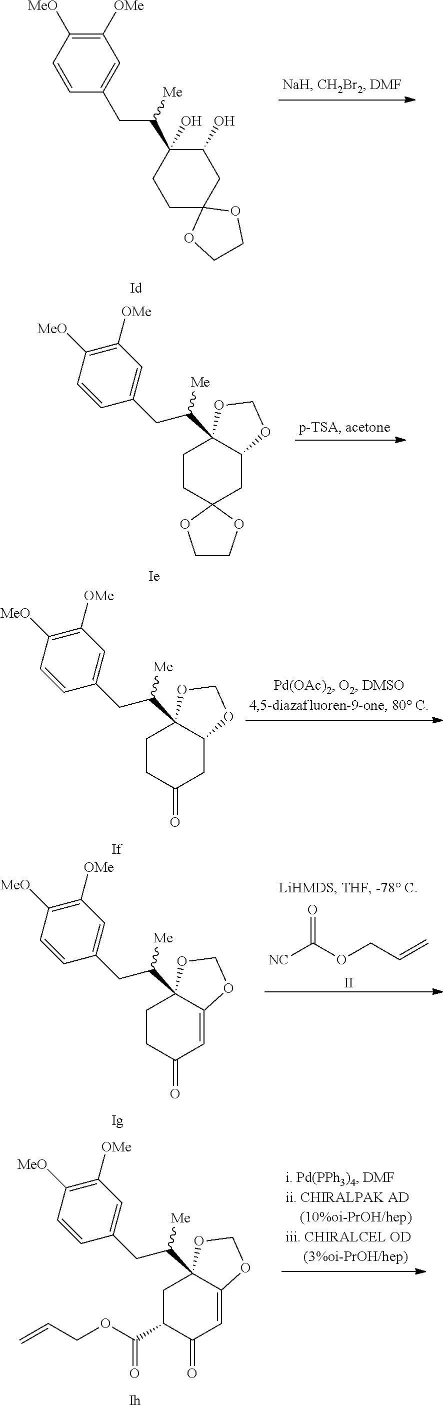

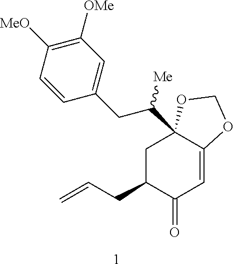 [SMILES: C=CC[C@H]1C[C@]2(C(C)Cc3ccc(OC)c(OC)c3)OCOC2=CC1=O ; "1"]

Step 1:

A mixture of I (117.0 g, 590 mmol), 4-iodo-1,2-dimethoxybenzene (85.0 g, 590 mmol), Pd(OAc)₂ (13.2 g, 59 mmol) and tri(o-tolyl)phosphine (36.0 g, 118 mmol) in triethylamine (3 L) was heated at 90° C. for 12 h. The mixture was cooled to room temperature and diluted with ethyl acetate (3 L). The organic phase was washed with brine, dried (MgSO₄), and concentrated in vacuo to dryness. The crude product was purified by silica gel chromatography (0 to 40% ethyl acetate/hexanes) to provide (1a) as a brown oil (87.0 g, 73% based on the recovered starting material). ¹H NMR (300 MHz, CDCl₃): δ 6.85 (s, 2H), 6.79 (s, 1H), 6.64 (s, 1H), 3.98-3.93 (m, 4H), 3.88 (s, 3H), 3.86 (s, 3H), 2.10-2.00 (m, 4H), 1.81 (s, 3H), 1.68 (d, J=12.9 Hz, 4H), 1.17 (s, 1H).

Step 2:

To a solution of 1a (32.0 g, 100.0 mmol) in 1000 mL of MeOH was added Pd(OH)₂/C (20 wt. % Pd on carbon, 6.4 g) under a nitrogen atmosphere. The mixture was stirred under one atmosphere of hydrogen for 1.5 h. The reaction mixture was filtered through a pad of celite and the filter cake was washed with ethyl acetate (500 mL). The filtrate was concentrated in vacuo to provide 1b as a colorless oil (28.0 g, 88%). The crude product was used without further purification. ¹H NMR (500 MHz, CDCl₃): δ 6.79 (d, J=8.0 Hz, 1H), 6.70 (dd, J=8.0, 2.0 Hz, 2H), 3.99-3.93 (m, 4H), 3.86 (s, 3H), 3.85 (s, 3H), 3.04 (dd, J=13.0, 3.0 Hz, 1H), 2.15 (t, J=11.0 Hz, 1H), 1.97-1.92 (m, 2H), 1.86-1.77 (m, 2H), 1.75-1.63 (m, 5H), 0.83 (d, J=7.0 Hz, 3H).

Step 3:

To a solution of 1b (28.0 g, 91.5 mmol) in pyridine (180 mL) was added thionyl chloride (13.3 mL, 183.0 mmol) at 0-5° C. The mixture was stirred at 5° C. for 2 h. Most of the pyridine was removed at reduced pressure. The residue was diluted with ethyl acetate (500 mL) and H₂O (100 mL). The organic phase was washed with brine, dried (MgSO₄), and concentrated in vacuo to dryness. The crude product was purified by silica gel chromatography (0 to 40% ethyl acetate/hexanes) to provide 1c as a brown oil (21.0 g, 80%). ¹H NMR (500 MHz, CDCl₃): δ 6.77 (dd, J=6.5, 2.0 Hz, 1H), 6.66 (dd, J=6.5, 2.0 Hz, 2H), 5.26 (d, J=3.5 Hz, 1H), 3.97 (d, J=3.5 Hz, 4H), 3.86 (s, 3H), 3.85 (s, 3H), 2.74 (dd, J=13.5, 6.5 Hz, 1H), 2.45-2.41 (m, 1H), 2.33-2.29 (m, 1H), 2.23-2.21 (m, 4H), 1.77-1.74 (m, 2H), 0.98 (d, J=6.5 Hz, 3H).

Step 4:

To a mixture of AD-mix-β (132 g) in t-BuOH (480 mL) and H₂O (480 mL) was added a solution of 1c (32.0 g, 100.0 mmol) in an ice bath. Methylanesulfonamide (29.0 g, 300.0 mmol) was added. The reaction mixture was stirred at room temperature for 7 d. The reaction mixture was diluted with MTBE (1000 mL). The organic phase was washed with saturated Na₂S₂O₅, brine, dried (MgSO₄) and concentrated in vacuo to dryness. The crude product was purified by silica gel chromatography (0 to 40% ethyl acetate/hexanes) to provide the mixture of diastereomers 1d as a brown oil (23.4 g, 67%). ¹H NMR (500 MHz, CDCl₃): δ 6.79 (d, J=8.0 Hz, 1H), 6.71 (m, 2H), 3.99-3.94 (m, 5H), 3.87 (s, 3H), 3.85 (s, 3H), 3.11 (s, 0.6H), 3.05 (dd, J=13.5, 3.5 Hz, 0.4 H), 2.93 (dd, J=13.5, 3.5 Hz, 0.5H), 2.25-2.20 (m, 2H), 2.13-1.95 (m, 3H), 1.89-1.62 (m, 4H), 1.09 (s, 0.4H), 0.87 (t, J=7.0 Hz, 3H).

Step 5:

To a solution of 1d (14.8 g, 44.3 mmol) in 110 mL of DMF was added NaH (60% dispersion in mineral oil, 5.0 g, 124.1 mmol) in an ice bath under a nitrogen atmosphere. After 1 h, dibromomethane (4.0 mL, 57.6 mmol) was added and the reaction mixture was stirred at room temperature for 12 h. The reaction was diluted with ethyl acetate (1000 mL) and washed with H₂O (100 mL). The organic phase was washed with brine, dried (MgSO₄), and concentrated in vacuo to dryness. The crude product was purified by silica gel chromatography (0 to 40% ethyl acetate/hexanes) to provide 1e as an off-white solid (680 mg, 82% based on the recovered starting material). ¹H NMR (500 MHz, CDCl₃): 6 6.79 (d, J=8.0 Hz, 1H), 6.69-6.65 (m, 2H), 5.19 (0.3H), 5.16 (s, 0.7H), 4.94 (s, 0.3H), 4.89 (s, 0.7H), 4.20-3.94 (m, 4H), 3.87 (s, 3H), 3.86 (s, 3H), 3.09 (d, J=14.0, 3.0 Hz, 0.3H), 2.80 (dd, J=14.0, 3.0 Hz, 0.7H), 2.18-2.02 (m, 2H), 1.98-1.63 (m, 7H), 0.89 (d, J=6.5 Hz, 2.1H), 0.81 (d, J=7.0 Hz, 0.9H).

Step 6:

To a solution of 1e (5.8 g, 16.0 mmol) in 300 mL of acetone wad added p-toluenesulfonic acid monohydrate (304 mg, 1.6 mmol). The reaction mixture was stirred at room temperature for 12 h. The acetone was removed under reduced pressure and the residue was purified by silica gel chromatography (0 to 40% ethyl acetate/hexanes) to provide 1f as a colorless oil (4.1 g, 80%). ¹H NMR (500 MHz, CD₃OD): δ 6.88 (d, J=21.5 Hz, 1H), 6.81-6.77 (m, 2H), 5.12 (s, 0.3H), 5.08 (s, 0.7H), 4.87 (s, 0.3H), 4.82 (s, 0.7H), 4.43 (t, J=2.5 Hz, 0.7H), 4.25 (t, J=2.5 Hz, 0.3Hz), 3.84 (s, 3H), 3.83 (s, 3H), 3.21 (dd, J=15.0, 3.0 Hz, 0.3H), 2.85 (dd, J=15.0, 3.0 Hz, 0.7H), 2.72-2.66 (m, 2H), 2.46-2.42 (m, 1H), 2.30-2.24 (m, 1H), 2.18-1.90 (m, 3H), 0.96 (d, J=6.5 Hz, 2.1H), 0.88 (d, J=6.5 Hz, 0.9H).

Step 7:

A mixture of 1f (1.0 g, 3.12 mmol), Pd(OAc)₂ (350 mg, 1.56 mmol) and 4,5-diazafluoren-9-one (278 mg, 1.56 mmol) in 40 mL of DMSO was heated to 80° C. under one atmosphere of oxygen for 6 h. The reaction mixture was cooled to room temperature and diluted with ethyl acetate (400 mL). The organic phase was washed with brine, dried (MgSO₄), and concentrated to dryness under reduced pressure. The crude residue was purified by chromatography (0 to 40% ethyl acetate/hexanes) to provide 1g as a white solid (670 mg, 67%). ¹H NMR (500 MHz, CD₃OD): δ 6.87 (d, J=8.0 Hz, 1H), 6.80-6.62 (m, 2H), 5.71 (s, 0.3H), 5.69 (s, 0.7H), 5,65 (s, 0.3H), 5.62 (s, 0.7H), 5.48 (s, 0.3H), 5.44 (s, 0.7H), 3.81 (s, 3H), 3.79 (s, 3H), 3.03 (dd, J=13.5, 4.0 Hz, 0.7H), 2.86 (dd, J=13.0, 4.0 Hz, 0.3H), 2.60-2.56 (m, 1H), 2.50-2.38 (m, 3H), 2.27-2.20 (m, 1H), 2.03-1.96 (m, 1H), 0.99 (d, J=7.0 Hz, 0.9H), 0.94 (d, J=7.0 Hz, 2.1H).

Step 8:

To a solution of 1g (1.8 g, 5.66 mmol) in THF (80 mL) was added LiHMDS (1.0M solution in THF, 8.5 mL, 8.5 mmol) at -78° C. under a nitrogen atmosphere. The reaction mixture was stirred at −78° C. for 30 min. Allyl cyanoformate II (942 mg, 8.49 mmol) was added. The reaction mixture was allowed to warm to 0° C. over 2 h. The reaction was quenched with H₂O and extracted with ethyl acetate (200 mL). The organic phase was washed with brine, dried (MgSO₄), and concentrated to dryness under reduced pressure. The residue was purified by silica gel chromatography (0 to 40% ethyl acetate/hexanes) to provide a mixture of isomers 1h as a brown gum (1.8 g, 82%). ¹H NMR (500 MHz, CD₃OD): δ 6.88-6.85 (m, 2H), 6.78 (d, J=7.5 Hz, 1H), 5.94-5.90 (m, 1H), 5.73 (s, 1H), 5.67 (s, 1H), 5.50 (s, 1H), 5.35 (dd, J=14.0, 3.0 Hz, 1H), 5.24 (d, J=10.5 Hz, 1H), 4.63 (d, J=2.5 Hz, 2H), 3.84 (s, 3H), 3.80 (s, 3H), 3.60 (dd, J=12.5, 5.0 Hz, 1H), 3.03 (dd, J=14.0, 5.0 Hz, 1H), 2.73 (dd, J=12.5, 5.0 Hz, 1H), 2.55 (dd, J=13.5, 9.0 Hz, 1H), 2.30-2.28 (m, 1H), 2.27 (t, J=12.5 Hz, 1H), 0.98 (d, J=7.0 Hz, 3H).

Step 9:

To a solution of 1h (4.9 g, 12.1 mmol) in DMF (120 mL) was added Pd(PPh₃)₄ (1.4 g, 1.21 mmol) under a nitrogen atmosphere. The reaction mixture was stirred at room temperature for 1 h, and then diluted with ethyl acetate (200 mL). The organic phase was washed with brine, dried (MgSO₄), and concentrated to dryness under reduced pressure. The residue was purified by silica gel chromatography (0 to 30% ethyl acetate/hexanes) to provide a 78:22 mixture of cis-isomer and trans-isomer 1 (3.8 g, 88%). The mixture was separated by CHIRALPAK AD (10% i-PrOH/heptane) to provide the enriched enantiomer 1 (1.5 g) with 63.4% ee. The enriched enantiomer (1.0 g) was further separated by chromatography on CHIRALCEL OD (3% i-PrOH/heptane) to provide 1 as a colorless oil (680 mg).

Compound 1 ((6S,7aR)-6-Allyl-7a-((S)-1-(3,4-dimethoxyphenyl)propan-2-yl)-7,7a-dihydrobenzo[d][1,3]dioxol-5(6H)-one). ¹H NMR (500 MHz, CD₃OD): δ 6.89 (d, J=8.0 Hz, 1H), 6.76 (s, 1H), 6.74 (dd, J=8.0, 2.0 Hz, 1H), 5.94-5.85 (m, 1H), 5.67 (s, 1H), 5.60 (s, 1H), 5.49 (s, 1H), 5.18 (dd, J=17.0, 10.5 Hz, 2H), 3.81 (s, 3H), 3.80 (s, 3H), 3.11 (dd, J=14.0, 4.0 Hz, 1H), 2.91 (dd, J=15.0, 1.5 Hz, 1H), 2.71 (d, J=16.0 Hz, 1H), 2.62 (t, J=3.5 Hz, 1H), 2.46 (t, J=11.0 Hz, 1H), 2.29-2.24 (m, 1H), 2.14 (dd, J=14.0, 9.5 Hz, 1H), 2.00-1.97 (m, 1H), 0.86 (d, J=7.0 Hz, 3H). ESI MS m/z 359 [M+H]⁺.

All references, including without limitation all papers, publications, presentations, texts, reports, manuscripts, brochures, internet postings, journal articles, periodicals, and the like, cited in this specification are hereby incorporated by reference. The discussion of the references herein is intended merely to summarize the assertions made by their authors and no admission is made that any reference constitutes prior art. The inventors reserve the right to challenge the accuracy and pertinence of the cited references.

It is intended that all patentable subject matter disclosed herein be claimed and that no such patentable subject matter be dedicated to the public. Thus, it is intended that the claims be read broadly in light of that intent. In addition, unless it is otherwise clear to the contrary from the context, it is intended that all references to “a” and “an” and subsequent corresponding references to “the” referring back to the antecedent basis denoted by “a” or “an” are to be read broadly in the sense of “at least one.” Similarly, unless it is otherwise clear to the contrary from the context, the word “or,” when used with respect to alternative named elements is intended to be read broadly to mean, in the alternative, any one of the named elements, any subset of the named elements or all of the named elements.

In view of the above, it will be seen that the several advantages of the invention are achieved and other advantageous results obtained. It should be understood that the aforementioned embodiments are for exemplary purposes only and are merely illustrative of the many possible specific embodiments that can represent applications of the principles of the invention. Thus, as various changes could be made in the above methods and compositions without departing from the scope of the invention, it is intended that all matter contained in the above description as shown in the accompanying drawings shall be interpreted as illustrative and not in a limiting sense.

Moreover, one of ordinary skill in the art can make various changes and modifications to the invention to adapt it to various usages and conditions, including those not specifically laid out herein, without departing from the spirit and scope of this invention. Accordingly, those changes and modifications are properly, equitably, and intended to be, within the full range of equivalents of the invention disclosed and described herein.

1. (canceled)

2. (canceled)

3. (canceled)

4. (canceled)

5. (canceled)

6. (canceled)

7. A method to inhibit tumor cells comprising the step of:
administering a therapeutically effective amount of a compound or stereoisomers thereof, according to formula:

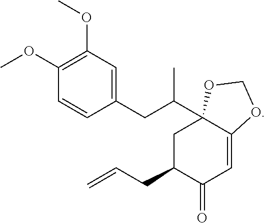 [SMILES: C=CC[C@H]1C[C@]2(C(C)Cc3ccc(OC)c(OC)c3)OCOC2=CC1=O]

8. A method to inhibit tumor cells comprising the step of:
administering a therapeutically effective amount of 6-allyl-7,7a-dihydro-7a-(1-(3,4-dimethoxyphenyl)propan-2-yl)benzo[d][1,3]dioxol-5(6H)-one.
